(19) United States
(12) Patent Application Publication
(10) Pub. No.: US 2024/0344011 A1
(43) Pub. Date: Oct. 17, 2024

(54) CELL COUNTING METHOD AND METHOD FOR DETERMINING THE EFFICACY OF A DRUG CANDIDATE

(71) Applicant: ONCOMEDICS, Limoges (FR)

(72) Inventors: Laurent NOYON, Limoges (FR); Thomas PERRAUDIN, Limoges (FR); Christophe LAUTRETTE, Limoges (FR); Stéphanie GIRAUD, Limoges (FR)

(73) Assignee: ONCOMEDICS, Limoges (FR)

(21) Appl. No.: 18/294,778

(22) PCT Filed: Aug. 4, 2022
(86) PCT No.: PCT/EP2022/071934

§ 371 Date: Feb. 2, 2024

(30) Foreign Application Priority Data
Aug. 4, 2021 (EP) 21306091.6

(51) Int. Cl. C12M 1/34; C12M 1/36; G01N 33/50

(52) U.S. Cl. CPC C12M 41/36; C12M 41/48; G01N 33/5011

(57) ABSTRACT

A computer implemented cell-counting method including using a computer to perform the steps of applying a predetermined image processing sequence to a sample sub-image to obtain a processed sub-image, the sample sub-image being extracted from a color sample image of a sample including cells of a subject, the image processing sequence including ga main processing series including a closing followed by an opening of the sample sub-image; and for each cluster of adjoining pixels of the processed sub-image: computing a corresponding relevancy score based on a value of at least one predetermined feature of the cluster; and determining that the cluster corresponds to a cell if the computed relevancy score belongs to a predetermined range.

### FIELD OF INVENTION

The present invention concerns a cell-counting method.

The invention also relates to a cell-counting device and to a method for determining the efficacy of a drug candidate or a drug association based on said cell-counting method.

The invention applies to the field of chemosensitivity assays.

### BACKGROUND OF INVENTION

Chemosensitivity assays, generally termed functional assays, have been developed for several decades. Assay procedures look at different endpoints, which all share the common feature of being measured on ex vivo models, either whole/minced patient tissue samples, or primary cultures derived from these.

During such assays, a sample (or a culture) comprising cancer cells is contacted with a drug candidate or a drug association candidate. The resulting sample is then contacted with both a colored label specific for dead cells and a colored label specific for living cells. Then, an operator individually counts the dead cells and the living cells, and it is concluded that the drug candidate is efficient against cancer if the ratio between, on the one hand, a first fraction of dead cells with respect to living cells in the sample that has been contacted with the drug candidate or the drug association candidate, and, on the other hand, a second fraction of dead cells with respect to living cells in a similar sample which has not been contacted with the drug candidate or the drug association candidate, is greater than a predetermined positivity threshold.

Alternatively, it is concluded that the drug candidate or the drug association is efficient against cancer if the fraction of dead cells with respect to living cells in the treated sample is greater than a predetermined positivity threshold.

However, such method is not satisfactory.

Indeed, individually counting cells can be very cumbersome and time-consuming, which, besides an obvious loss of time, may result in errors.

A purpose of the invention is therefore to provide a method for counting cells that is automated, efficient and reliable, while being simple.

### SUMMARY

To this end, the present invention is a cell-counting method of the aforementioned type, the cell-counting method including using a processing unit to perform the steps:
applying a predetermined image processing sequence to a sample sub-image to obtain a processed sub-image, the sample sub-image being extracted from a color sample image of a sample including cells of a subject, the image processing sequence comprising a main processing series including a closing followed by an opening of the sample sub-image; and
for each cluster of adjoining pixels of the processed sub-image:
   computing a corresponding relevancy score based on a value of at least one predetermined feature of the cluster; and
   determining that the cluster corresponds to a cell if the computed relevancy score belongs to a predetermined range.

In other words, the method comprises:
receiving the color sample image of the sample;
extracting a sample sub-image from said color sample image of the sample;
applying a predetermined image processing sequence to said sample sub-image to obtain a processed sub-image, said image processing sequence comprising a main processing series including a closing followed by an opening of the sample sub-image, so as to obtain a processed sub-image comprising at least one cluster of adjoining pixels; and
counting the cells in the sample by, for each cluster of the processed sub-image:
   computing a corresponding relevancy score based on a value of at least one predetermined feature of the cluster; and
   determining that the cluster corresponds to a cell if the computed relevancy score belongs to a predetermined range.

By “cluster of adjoining pixels”, refers to a continuous group of pixels having a relative intensity within a predetermined, preferably narrow, range of intensities. The term adjoining refers to the fact that pixels are connected if their edges touch, i.e., two adjoining pixels are part of the same cluster if their values are both in the same range of intensities and are connected along the horizontal or vertical direction. By contrast other neighboring pixels having a relative intensity outside said predetermined range or having no horizontal or vertical direction connection (i.e., edge contact) with at least one pixel of one cluster do not belong to said cluster of adjoining pixels.

Indeed, by performing such closing, neighboring pixels or pixel clusters that relate to a single cell, but that may appear as independent from one another on the acquired sample image (or sample sub-image) are reconnected in a simple way to form a pixel aggregate. Moreover, performing opening provides, to the pixel aggregate, a shape and a size that are similar to those of the actual cell to which the pixel aggregate relates. Such opening also reduces noise that may be caused by the closing, and that, for instance, may result in undesirable connections between neighboring pixel clusters.

Consequently, an accurate reconstruction of the cells in the sample sub-image is achieved. Furthermore, by computing a score for each cluster of pixels, proficient noise filtering is performed. Both results are attained in a manner that neither requires complex algorithms or nor necessitates extensive computational time.

According to other advantageous aspects of the invention, the method includes one or more of the following features, taken alone or in any possible combination:
the relevancy score is a function of at least one of:
   an area score based on a comparison between an area of the cluster with a predetermined reference area;
   a shape score based on a comparison between a shape of the cluster with a predetermined reference shape; and
   an absolute amplitude score depending on a mean amplitude of the cluster and on a distribution of the mean amplitude across the clusters of the processed sub-image;
the extracted sub-image corresponds to a predetermined color channel of the sample image;
the relevancy score is also a function of a relative amplitude score, the relative amplitude score being based on a comparison between the mean amplitude of the cluster in the processed sub-image and a mean amplitude of a cluster located at a same position in a reference sub-image extracted from the same sample image and corresponding to a color channel that is different from the color channel corresponding to said extracted sub-image;
the relevancy score is a weighted sum of at least two of the area score, the absolute amplitude score, the shape score and, preferably, the relative amplitude score;
the extracted sub-image is computed based on at least two color channels of the sample image;
the image processing sequence includes, prior to the main processing series, an adaptative thresholding, a threshold value associated to a given pixel being a function of an amplitude of neighboring pixels of said pixel;
the image processing sequence includes an additional processing series after the main processing series, the additional processing series comprising a closing followed by a segmentation, preferably a watershed segmentation;
the image processing sequence includes at least one ring filling after the main processing series, the ring filling comprising:
   detecting ring-shaped clusters of pixels;
   filling each detected ring-shaped cluster to transform said ring-shaped cluster into a solid pixel cluster;
the image processing sequence includes at least one cluster removing for removing clusters having an area lower than a predetermined area threshold and/or removing clusters having a mean amplitude lower than a predetermined amplitude threshold.

According to one embodiment, the method of the present invention is adapted for determining the efficacy of a drug candidate or of a drug association against cancer. In this embodiment, the sample includes cancerous cells and the extracted sub-image corresponds at least to a color channel associated to a spectral range where the colored label specific for living cells transmits or emits light and to the color channel associated to the spectral range where the colored label specific for dead cells transmits or emits light, so as to obtain the count the total number of cells in the color sample image. In this embodiment, method further comprises:
receiving a color sample image of a treated sample, wherein the sample including cancerous cells have been cultivated, put in contact with the drug candidate or the drug association to obtain the treated sample and said treated sample has been put in contact at least with a colored label specific for dead cells and a colored label specific for living cells;
extracting a treated sample sub-image from the color sample image of the treated sample, said treated sample sub-image corresponding to a color channel associated to a spectral range where the colored label specific for dead cells transmits or emits light;
applying the predetermined image processing sequence to the treated sample sub-image and counting cells in the treated sample sub-image, so as to obtain the number of dead cells in the treated sample;
concluding that the drug candidate or the drug association is efficient against the cancer if a ratio between, on the one hand, a fraction of dead cells with respect to living cells in the color sample image, and, on the other hand, a control fraction, is greater than a predetermined positivity threshold;
the control fraction being equal to a fraction of dead cells with respect to living cells in a control sample of the subject which includes cancerous cells and which has not been contacted with the drug candidate or the drug association candidate.

The invention also relates to a cell-counting device comprising a processing unit configured to:
apply a predetermined image processing sequence to a sample sub-image to obtain a processed sub-image, the sample sub-image being extracted from a color sample image of a sample including cells of a subject, the image processing sequence comprising a main processing series including a closing followed by an opening of the sample sub-image; and
for each cluster of adjoining pixels of the processed sub-image:
   compute a corresponding relevancy score based on a value of at least one predetermined feature of the cluster; and
   determine that the cluster corresponds to a cell if the computed relevancy score belongs to a predetermined range.

The invention also relates to a method for determining the efficacy of a drug candidate or of a drug association against cancer, the method including:
providing a sample previously obtained from a subject, the sample including cancerous cells;
cultivating the sample;
contacting the cultivated sample with the drug candidate or the drug association to obtain a treated sample;
contacting the treated sample at least with a colored label specific for dead cells and a colored label specific for living cells;
performing the cell counting method as previously defined to count the number of dead cells in a color sample image of the treated sample, the corresponding extracted sub-image corresponding to a color channel associated to a spectral range where the colored label specific for dead cells transmits or emits light;
performing the cell counting method as previously defined to count the total number of cells in the color sample image, the corresponding extracted sub-image corresponding at least to a color channel associated to a spectral range where the colored label specific for living cells transmits or emits light and to the color channel associated to the spectral range where the colored label specific for dead cells transmits or emits light;
concluding that the drug candidate or the drug association is efficient against the cancer if a ratio between, on the one hand, a fraction of dead cells with respect to living cells in the color sample image, and, on the other hand, a control fraction, is greater than a predetermined positivity threshold;
   the control fraction being equal to a fraction of dead cells with respect to living cells in a control sample of the patient which includes cancerous cells and which has not been contacted with the drug candidate or the drug association candidate.

### BRIEF DESCRIPTION OF THE DRAWINGS

FIG. 1 is a schematic view of a cell-counting device according to the invention;

FIG. 2 is a flowchart of a cell-counting method according to the invention; and

FIG. 3 is a detailed flowchart of a processing step of the method of FIG. 2.

### DETAILED DESCRIPTION

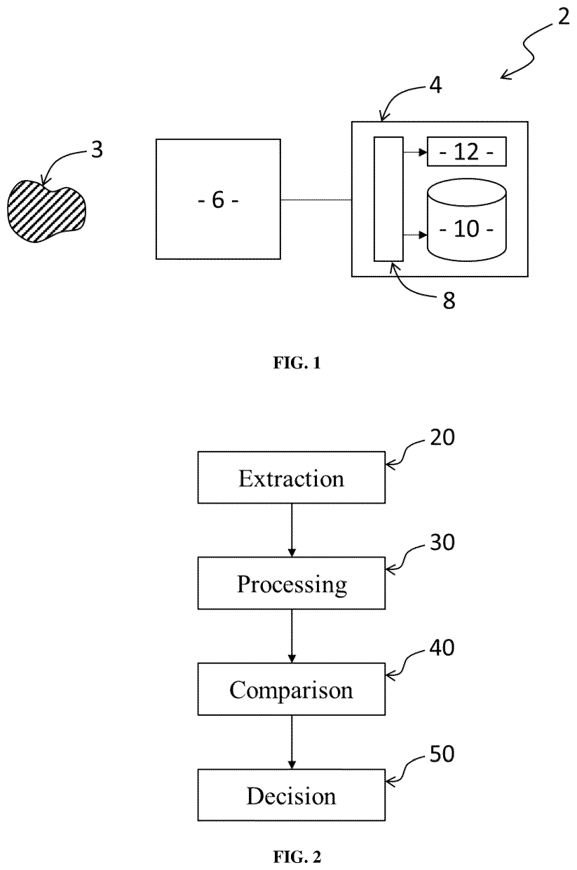

A cell-counting device 2 according to the invention is shown on FIG. 1.

The cell-counting device 2 is intended for automatically counting cells in a sample 3 including cells of a subject, for instance to determine the efficacy against cancer of a drug candidate or a drug association.

Such sample 3 is preferably a test sample including cancerous cells, which has been cultivated, contacted with the drug candidate or the drug association, then contacted with at least two colored labels. Such sample may also be a control sample prepared in a similar as the test sample, albeit without being contacted with the drug candidate or the drug association. Preferably, the sample is deposited on a microscope slide.

Said colored labels include a colored label specific for dead cells (also called “first label”) and a colored label specific for living cells (also called “second label”). Preferably, the sample is also contacted with a colored label able to color both dead and living cells (also called “third label”).

The first label is able to attach exclusively to dead cells in order to allow their detection. This first label is further designed to transmit or emit light (for instance a fluorescence light) in a first spectral range, for instance in the red range of the visible light spectrum. For instance, the first label is ethidium homodimer-1, which has a fluorescence peak at 617 nm (nanometer).

The second label is able to attach exclusively to living cells in order to allow their detection, for instance by being metabolized by such cells. This second label is further designed to transmit or emit light (for instance a fluorescence light) in a second spectral range, distinct from the first spectral range, for instance in the green range of the visible light spectrum. For instance, the second label is calcein AM (also known as “calcein acetoxymethyl”), which has a fluorescence peak at 517 nm.

The third label is able to bind with both dead cells and living cells, so that all the cells, either living or dead, can be optically identified. Preferably, the third label is able to bind to all cells that have been previously permeabilized, for instance by using formaldehyde which creates pores in the cellular membrane, thus allowing the third label to migrate through it. This third label is further designed to transmit or emit light (for instance a fluorescence light) in a third spectral range, distinct from the first spectral range and the second spectral range, for instance in the blue range of the visible light spectrum. For instance, the third label is DAPI (also known as “4′,6-diamidino-2-phenylindole”), which has a fluorescence peak at 461 nm.

The cell-counting device 2 includes a processing unit 4, and, optionally, a camera 6 connected to the processing unit 4.

The camera 6 may be any camera suitable for acquiring at least one color image (hereinafter, “sample image”) of the sample 3, and to transfer each acquired sample image to the processing unit 4. The camera 6 is preferably coupled to a microscope to acquire the sample image of the sample deposited on the aforementioned microscope slide.

The processing unit 4 is configured to count cells (that is to say dead and/or living cells) in the sample based on the corresponding sample image.

According to the invention, the expression “processing unit” should not be construed to be restricted to hardware capable of executing software, and refers in a general way to a processing device, which can for example include a microprocessor, an integrated circuit, or a programmable logic device (PLD). The processing unit 4 may also encompass one or more Graphics Processing Units (GPU) or Tensor Processing Units (TSU), whether exploited for computer graphics and image processing or other functions. Additionally, the instructions and/or data enabling to perform associated and/or resulting functionalities may be stored on any processor-readable medium such as, e.g., an integrated circuit, a hard disk, a CD (Compact Disc), an optical disc such as a DVD (Digital Versatile Disc), a RAM (Random-Access Memory) or a ROM (Read-Only Memory). Instructions may be notably stored in hardware, software, firmware or in any combination thereof.

As an exemplary embodiment, the processing unit 4 comprises an input/output interface 8 (hereinafter “I/O interface”), a memory 10 and a microprocessor 12.

The I/O interface 8 is configured to receive the acquired sample image and to output at least one result of a processing performed based on the received sample image. Moreover, the memory 10 is configured to store the received sample image, as well as a software for processing said received sample image. Finally, the microprocessor 12 is configured to execute the software stored in the memory 10 to process the sample image in order to determine a number of cells in the sample (or, more precisely, a number of cells in the sample image).

As shown in FIG. 2, the processing unit 4 is configured to perform an extraction step 20, a processing step 30, a comparison step 40 and a decision step 50.

More precisely, the processing unit 4 is configured to extract, during the extraction step 20, a first sample sub-image from the color sample image.

Advantageously, the extracted first sample sub-image corresponds to a predetermined color channel of the sample image, among the red channel, the green channel and the blue channel.

For instance, the extracted first sample sub-image corresponds to the color channel that is associated to the wavelength range which includes the wavelength at which the first label (which is specific to dead cells) is designed to transmit or emit a maximum amount of light. As an example, if the first label is designed to transmit or emit light in the red range of the visible light spectrum, then the first sample sub-image corresponds to the red channel of the sample image. In this case, the first sample sub-image only shows dead cells.

Preferably, the processing unit 4 is further configured to extract, during the extraction step 20, a reference sub-image from the same sample image as the first sample sub-image. Such reference sub-image corresponds to a color channel that is different from the color channel associated to the first sample sub-image.

For instance, if the second label, which is specific to living cells, is designed to transmit or emit light in the green range of the visible light spectrum, then the extracted reference sub-image corresponds to the green channel of the sample image. In this case, the reference sub-image only shows living cells.

Preferably, the processing unit 4 is also configured to extract, during the extraction step 20, a second sample sub-image from the color sample image, based on at least two color channels of the sample image, preferably three color channels of the sample image.

For instance, the extracted second sample sub-image corresponds to a result of merging all the color channels of the sample image. In this case, the extracted second sample sub-image shows both dead cells and living cells.

Advantageously, the processing unit 4 is configured to adjust a brightness (i.e., a lightness) level of each color channel of the sample image, prior to extracting the first sample sub-image, the second sample sub-image and/or the reference sub-image. Such operation aims at providing a background that has the lowest possible value.

The processing unit 4 is also configured to apply, during the processing step 30, and after the extraction step 20, a predetermined image processing sequence to the first sample sub-image to obtain a first processed sub-image.

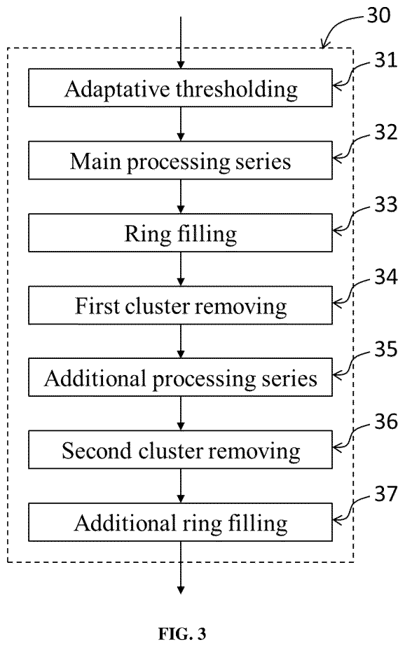

The image processing sequence comprises at least one processing routine. More precisely, as shown in FIG. 3, the image processing sequence comprises, the following routines in the following order: an adaptative thresholding 31, a main processing series 32, a ring filling 33, a first cluster removing 34, an additional processing series 35, a second cluster removing 36 and an additional ring filling 37. However, each of the adaptative thresholding 31, the ring filling 33, the first cluster removing 34, the additional processing series 35, the second cluster removing 36 and the additional ring filling 37 is optional.

Throughout the description of the routines of the image processing sequence, the expression “sample sub-image” used to designate the input of any given routine shall actually refer to an image resulting from the implementation of a previous routine of the image processing sequence.

To perform the adaptative thresholding 31, the processing unit 4 is configured to compare a value (that is, the value of its brightness, i.e., intensity value of a pixel, also called in the present description “amplitude”) of each pixel of the sample sub-image to a threshold value associated to said pixel, and to put said pixel in the background (for instance by assigning “0” to its value, i.e., its amplitude) if the value of the pixel is less than the corresponding threshold value. For any given pixel, the corresponding threshold value is a function of a neighbor pixels' amplitude calculated from the amplitudes of its neighboring pixels. The neighboring pixels of one considered pixel refers to a group of pixels that are not adjoining to it, i.e., pixels that have no edge or corners in contact with the considered pixel. The neighboring pixels may comprise therefore all the other pixels of the processed sub-image, except for the adjoining pixels of the considered pixel. Alternatively, the neighboring pixels may comprise a group of pixels located at a predefined distance or located within a range of distances from the considered pixel. For example, the neighboring pixels may comprise the pixels of the processed sub-image having a distance superior to a first predefined distance d and inferior to a second predefined distance D. The distance may be given in dots par inch, pixels per inch or millimeters and the like.

This is advantageous, as such feature allows to counterbalance potential brightness inconsistencies across the sample sub-image which are due to, for instance, overexposure.

The main processing series 32 includes a closing followed by an opening of the sample sub-image. Consequently, to perform the main processing series 32, the processing unit 4 is configured to perform a closing followed by an opening of the sample sub-image.

In the context of the invention, by “closing”, it is meant an erosion of a dilation of an image. Moreover, in the context of the invention, by “opening”, it is meant a dilation of an erosion of an image.

For instance, the closing and/or the opening is performed using a structuring element having an elliptical shape. The size and the parameters of such structuring element may depend on the type of operation being performed (opening or closing) and the type of sample sub-image being processed (first sample sub-image or second sample sub-image).

The implementation of the main processing series 32 is advantageous. Indeed, by performing closing, neighboring pixels or pixel clusters that relate to a single cell, but appear as independent from one another on the acquired sample image (or sample sub-image) are reconnected in a simple way to form a pixel aggregate. Moreover, performing opening provides, to the pixel aggregate, a shape and a size that are similar to those of the actual cell to which the pixel aggregate relates. Such opening also reduces noise that may be caused by the closing, and that, for instance, may result in undesirable connections between neighboring clusters of pixels.

To perform the ring filling 33, the processing unit 4 is configured to detect ring-shaped clusters of pixels in the sample sub-image. The processing unit 4 is further configured to fill each detected ring-shaped cluster to transform said ring-shaped cluster into a solid cluster of pixels.

To perform the first cluster removing 34, the processing unit 4 is configured to determine an area of each pixel cluster, and to remove clusters having an area lower than a predetermined area threshold. Alternatively, or in addition, the processing unit 4 is configured to determine a mean amplitude of each pixel cluster, and to remove clusters having a mean amplitude lower than a predetermined amplitude threshold. The mean amplitude of each pixel cluster (also “mean cluster amplitude”) is obtained as the mean of the amplitude values of the pixels belonging to the cluster.

Such routine is advantageous, as it allows to remove pixel clusters that are actually artefacts, thus reducing computational time when performing the following routines.

The additional processing series 35 includes a closing followed by a segmentation of the sample sub-image. Consequently, to perform the additional processing series 35, the processing unit 4 is configured to perform a closing followed by a segmentation of the sample sub-image.

This is advantageous, as the segmentation allows to distinguish between cells (i.e., pixel clusters) that join at their boundary, therefore enhancing the relevancy of the following steps.

Preferably, the segmentation is a watershed segmentation.

Moreover, the second cluster removing 36 and the additional ring filling 37 are identical to the first cluster removing 34 and the ring filling 33, respectively.

The processing unit 4 may also be configured to apply, during the processing step 30, the predetermined image processing sequence to the second sample sub-image to obtain a second processed sub-image, and/or to the reference sub-image to further modify features of the reference sub-image.

The processing unit 4 is also configured to compute, during the comparison step 40, and after the processing step 30, a relevancy score for each cluster of adjoining pixels of the obtained first processed sub-image.

More precisely, the processing unit 4 is configured to compute, during such comparison step 40, the relevancy score for each cluster of adjoining pixels of the first processed sub-image based on a value of at least one predetermined feature of the cluster.

Such feature may be an area of the cluster or a mean amplitude of the cluster. The term “mean amplitude of a cluster” refers to the mean value of the intensity values (i.e., amplitudes) of the pixels comprised in said cluster. In this case, the relevancy score is a function of at least one of an area score based on the area of the cluster and an absolute amplitude score based on the mean amplitude of the cluster, and a shape score based on the shape of the cluster.

More precisely, the area score is based on a comparison between the area of the cluster with a predetermined reference area. For instance, for a given cluster, the area score is based on a ratio between the area of the cluster with a predetermined reference area. Such area score may be comprised between 0 and 1.

Moreover, the absolute amplitude score depends on the mean amplitude of the cluster and on a distribution of the mean amplitude across the clusters of the first processed sub-image.

For instance, for a given cluster, the absolute amplitude score is a function of a comparison between the mean amplitude of said cluster and a predetermined quantile of the mean amplitude distribution.

As an example, for a given cluster, the absolute amplitude score is equal to 1 if the mean amplitude of said cluster is greater that a predetermined decile of the mean amplitude distribution, and is equal to a ratio between the mean amplitude of said cluster and said predetermined decile otherwise.

More precisely, the shape score is based on a comparison between the shape of the cluster with a predetermined reference cell shape. For instance, for a given cluster, the most favorable values of the shape score are obtained if a dissimilarity between the shape of the cluster and the reference cell shape is minimal.

Alternatively, or in addition, the relevancy score may be a function of a relative amplitude score based on the mean amplitude of the cluster.

More precisely, the relative amplitude score is based on a comparison between the mean amplitude of the cluster in the first processed sub-image (hereinafter, “first amplitude”) and a mean amplitude of a cluster located at a same position in the reference sub-image (hereinafter, “second amplitude”).

In this case, the reference sub-image may have been previously processed through the implementation, by the processing unit 4, of the aforementioned processing step 30.

For instance, for a given cluster, the relative amplitude score is a function of a difference between, on the one hand, the first amplitude multiplied by a predetermined coefficient, and, on the other hand, the second amplitude.

Multiplying the first amplitude by the predetermined coefficient allows to account for the fact that the human eye perceives green in a more acute way than it does with red. Therefore, using the predetermined coefficient allows to mimic, in a very simple manner, the behavior of an experienced operator performing cell counting.

Preferably, the processing unit 4 is configured to compute the relevancy score as a weighted sum of at least two of the area score, the absolute amplitude score, the shape score and, preferably, the relative amplitude score.

Even more preferably, for any given cluster of pixels, if the second amplitude is very low compared to the first amplitude, and if the area of the cluster of pixels is lower than a predetermined minimal area, the processing unit 4 is configured to assign, to the relevancy score, a value that is outside a predetermined range.

The processing unit 4 is also configured to compute, during the comparison step 40, a relevancy score for each cluster of adjoining pixels of the obtained second processed sub-image, in a similar fashion as above. Nevertheless, in this case, for each cluster of pixels of the second processed sub-image, the relevancy score is only a function of at least one of the area score, the shape score and the absolute amplitude score.

The processing unit 4 is also configured to determine, during the decision step 50, and after the comparison step 40, a nature of each cluster of adjoining pixels of the first processed sub-image and/or the second processed sub-image.

More precisely, the processing unit is configured to determine, during the decision step 50, that the cluster corresponds to a cell if the computed relevancy score belongs to the predetermined range.

If the cluster corresponds to a cell, the processing unit is further configured to increment a number of dead cells indicator and/or a number of living cells indicator and/or a total number of cells indicator, associated to the sample 3.

The processing unit 4 is configured to implement the decision step 50 to the first processed sub-image (to identify and count the dead cells), but also to the second processed image (to identify both dead and living cells, and to count the total number of cells).

Moreover, the processing unit 4 is configured to conclude, during the decision step 50, that the drug candidate or the drug association is efficient against the cancer if a ratio between, on the one hand, the fraction of dead cells with respect to living cells in the sample 3 that has been contacted with the drug candidate or the drug association candidate, and, on the other hand, the fraction of dead cells with respect to living cells in a similar sample which has not been contacted with the drug candidate or the drug association candidate, is greater than a predetermined positivity threshold.

Alternatively, the fraction that is considered is the fraction of dead cells relative to the total number of cells.

The I/O interface 8 is configured to output the result(s) obtained by performing the decision step 50.

Operation of the cell-counting device 2 will now be disclosed.

The camera 6 acquires at least one sample image of a sample as described above, and transfers each acquired sample image to the processing unit 4.

Then, the processing unit 4 extracts, during the extraction step 20, the first sample sub-image from the color sample image.

Preferably, the processing unit 4 also extracts, during the extraction step 20, the reference sub-image and/or the second sample sub-image from the color sample image.

Then, during the processing step 30, the processing unit 4 applies the predetermined image processing sequence to the first sample sub-image to obtain the first processed sub-image.

Preferably, during the processing step 30, the processing unit 4 also applies the predetermined image processing sequence to the reference sub-image and/or the second sample sub-image to obtain the updated reference sub-image and/or the second processed sub-image, respectively.

Then, during the comparison step 40, the processing unit 4 computes the relevancy score for each cluster of adjoining pixels of the obtained first processed sub-image.

Preferably, during the comparison step 40, the processing unit 4 also computes the relevancy score for each cluster of adjoining pixels of the obtained second processed sub-image.

Then, during the decision step 50, the processing unit 4 determines whether each cluster of adjoining pixels of the first processed sub-image and/or the second processed sub-image corresponds to a cell or not.

If the cluster corresponds to a cell, the processing unit 4 increments a number of cells indicator.

Then, the processing unit 4 concludes, during the decision step 50, that the drug candidate or the drug association is efficient against the cancer if a ratio between, on the one hand, the fraction of dead cells with respect to living cells in the sample 3 that has been contacted with the drug candidate or the drug association candidate, and, on the other hand, the fraction of dead cells with respect to living cells in a similar sample which has not been contacted with the drug candidate or the drug association candidate, is greater than the predetermined positivity threshold.

This result is then output through the I/O interface 8.

1-13. (canceled)

14. A computer implemented method for counting cells in a color sample image of a sample comprising cells of a subject, said method including using a processing unit to perform:
receiving the color sample image of the sample;
extracting a sample sub-image from said color sample image of the sample;
applying a predetermined image processing sequence to said sample sub-image to obtain a processed sub-image, said image processing sequence comprising a main processing series including a closing followed by an opening of the sample sub-image, so as to obtain a processed sub-image comprising at least one cluster of adjoining pixels; and
counting the cells in the sample by, for each cluster of the processed sub-image:
computing a corresponding relevancy score based on a value of at least one predetermined feature of the cluster; and
determining that the cluster corresponds to a cell if the computed relevancy score belongs to a predetermined range.

15. The cell-counting method according to claim 14, wherein the relevancy score is a function of at least one of:
an area score based on a comparison between an area of the cluster with a predetermined reference area;
a shape score based on a comparison between a shape of the cluster with a predetermined reference shape; and
an absolute amplitude score depending on a mean amplitude of the cluster, calculated as a mean of an amplitude value of the pixels in said cluster, and on a distribution of the mean amplitude across the clusters of the processed sub-image.

16. The cell-counting method according to claim 14, wherein the extracted sub-image corresponds to a predetermined color channel of the sample image.

17. The cell-counting method according to claim 16, wherein the relevancy score is also a function of a relative amplitude score, the relative amplitude score being based on a comparison between the mean amplitude of the pixels in the cluster in the processed sub-image and a mean amplitude of the pixels comprised in a cluster located at a same position in a reference sub-image extracted from the same sample image and corresponding to a color channel that is different from the color channel corresponding to said extracted sub-image.

18. The cell-counting method according to claim 15, wherein the relevancy score is a weighted sum of at least two of the area score, the absolute amplitude score, the shape score.

19. The cell-counting method according to claim 15, wherein the relevancy score is a weighted sum of at least two of the area score, the absolute amplitude score, the shape score and the relative amplitude score.

20. The cell-counting method according to claim 14, wherein the extracted sub-image is computed based on at least two color channels of the sample image.

21. The cell-counting method according to claim 14, wherein the image processing sequence includes, prior to the main processing series, an adaptative thresholding, a threshold value associated to a given pixel being a function of an amplitude of neighboring pixels of said pixel.

22. The cell-counting method according to claim 14, wherein the image processing sequence includes an additional processing series after the main processing series, the additional processing series comprising a closing followed by a segmentation.

23. The cell-counting method according to claim 14, wherein the image processing sequence includes an additional processing series after the main processing series, the additional processing series comprising a closing followed by a watershed segmentation.

24. The cell-counting method according to claim 14, wherein the image processing sequence includes at least one ring filling after the main processing series, the ring filling comprising:
detecting ring-shaped clusters of pixels;
filling each detected ring-shaped cluster to transform said ring-shaped cluster into a solid pixel cluster.

25. The cell-counting method according to claim 14, wherein the image processing sequence includes at least one cluster removing for removing clusters having an area lower than a predetermined area threshold and/or removing clusters having a mean amplitude lower than a predetermined amplitude threshold.

26. The cell-counting method according to claim 14, for determining the efficacy of a drug candidate or of a drug association against cancer, wherein the sample includes cancerous cells; wherein the extracted sub-image corresponds at least to a color channel associated to a spectral range where the colored label specific for living cells transmits or emits light and to the color channel associated to the spectral range where the colored label specific for dead cells transmits or emits light, so as to obtain the count the total number of cells in the color sample image; said method further comprising:
receiving a color sample image of a treated sample, wherein the sample including cancerous cells have been cultivated, put in contact with the drug candidate or the drug association to obtain the treated sample and said treated sample has been put in contact at least with a colored label specific for dead cells and a colored label specific for living cells;
extracting a treated sample sub-image from the color sample image of the treated sample, said treated sample sub-image corresponding to a color channel associated to a spectral range where the colored label specific for dead cells transmits or emits light;
applying the predetermined image processing sequence to the treated sample sub-image and counting cells in the treated sample sub-image, so as to obtain the number of dead cells in the treated sample;
concluding that the drug candidate or the drug association is efficient against the cancer if a ratio between, on the one hand, a fraction of dead cells with respect to living cells in the color sample image, and, on the other hand, a control fraction, is greater than a predetermined positivity threshold;
the control fraction being equal to a fraction of dead cells with respect to living cells in a control sample of the subject which includes cancerous cells and which has not been contacted with the drug candidate or the drug association candidate.

27. A cell-counting device comprising a processing device configured to:
extract a sample sub-image from a color sample image of a sample including cells of a subject;
apply a predetermined image processing sequence to a sample sub-image to obtain a processed sub-image, said image processing sequence comprising a main processing series including a closing followed by an opening of the sample sub-image, so as to obtain a processed sub-image comprising at least one cluster of adjoining pixels; and
count the cells in the sample by, for each cluster of the processed sub-image:
compute a corresponding relevancy score based on a value of at least one predetermined feature of the cluster; and
determine that the cluster corresponds to a cell if the computed relevancy score belongs to a predetermined range.

28. A method for determining the efficacy of a drug candidate or of a drug association against cancer, the method including:
providing a sample previously obtained from a subject, the sample including cancerous cells;
cultivating the sample;
contacting the cultivated sample with the drug candidate or the drug association to obtain a treated sample;
contacting the treated sample at least with a colored label specific for dead cells and a colored label specific for living cells;
performing the cell counting method according to claim 14 to count the number of dead cells in a color sample image of the treated sample, the corresponding extracted sub-image corresponding to a color channel associated to a spectral range where the colored label specific for dead cells transmits or emits light;
performing said cell counting method to count the total number of cells in the color sample image, the corresponding extracted sub-image corresponding at least to a color channel associated to a spectral range where the colored label specific for living cells transmits or emits light and to the color channel associated to the spectral range where the colored label specific for dead cells transmits or emits light;
concluding that the drug candidate or the drug association is efficient against the cancer if a ratio between, on the one hand, a fraction of dead cells with respect to living cells in the color sample image, and, on the other hand, a control fraction, is greater than a predetermined positivity threshold;
the control fraction being equal to a fraction of dead cells with respect to living cells in a control sample of the patient which includes cancerous cells and which has not been contacted with the drug candidate or the drug association candidate.
